(19) United States
(12) Patent Application Publication
(10) Pub. No.: US 2025/0201385 A1
(43) Pub. Date: Jun. 19, 2025

(54) MEDICAL DATA EXCHANGE

(71) Applicant: IMAGEMOVERMD, INC., Madison, WI (US)

(72) Inventors: Gary Wendt, Madison, WI (US); Mike Courtney, Madison, WI (US); Mark Gehring, Madison, WI (US); Brett Young-Moxon, Madison, WI (US); Will Robus, Madison, WI (US); Kevin Houlihan, Madison, WI (US); K. Thomas Pickard, Danville, CA (US)

(21) Appl. No.: 18/957,320

(22) Filed: Nov. 22, 2024

Related U.S. Application Data
(63) Continuation of application No. 17/711,271, filed on Apr. 1, 2022, now abandoned.
(60) Provisional application No. 63/169,644, filed on Apr. 1, 2021.

(51) Int. Cl. G16H 30/20; G16H 10/60

(52) U.S. Cl. CPC G16H 30/20; G16H 10/60

(57) ABSTRACT

The present disclosure provides methods, systems, and non-transitory computer-readable media for transfer or exchange of medical data (e.g., medical images). The systems and methods allow seamless retrieval and secure transfer of remote medical data without additional authentication and minimal user input.

### CROSS-REFERENCE TO RELATED APPLICATIONS

This application is a continuation of U.S. application Ser. No. 17/711,271, filed Apr. 1, 2022, which claims the benefit of U.S. Provisional Application No. 63/169,644, filed Apr. 1, 2021, which are herein incorporated by reference in its entirety.

### FIELD

The present disclosure provides computer-implemented methods, systems, and non-transitory computer-readable media for peer-to-peer transfer or exchange of medical data (e.g., medical images).

### BACKGROUND

Patients commonly transition between health systems and within dedicated units within a single or shared health system. Image exchange allows the new institution or physician to be provided a full patient imaging history and reduces the need for duplicate examinations which can lead to additional costs, lost revenue, patient care delays, and, occasionally, decreased patient exposure to imaging agents, for example.

Currently, the most common image exchange mechanism is for the patient to physically deliver images from one unit or health system on a computer readable medium (e.g., CD, DVD, flash memory, such as thumb drive or flash memory card, or other similar physical media). Commercial systems for image transfer can use exchange of faxes or rely on a centralized, cloud-based image repository which pose concerns regarding control and accessibility of data. These existing systems generally look at transferring data from any patient at the remote health system, resulting in complex approval or authentication workflows. Furthermore, imagining standards (e.g., Digital Imaging and Communication in Medicine (DICOM)) which support encryption are not commonly supported or deployed. Thus, improved systems are needed.

### SUMMARY

This disclosure relates to computer-implemented methods and non-transitory computer-readable media for exchanging medical data, including medical imaging data, between computer systems. In some embodiments, the computer systems are connected in a peer to peer network. In some embodiments, the files may route through remote or cloud servers. In some embodiments, the computer systems determine the address or location of other computer systems using a centralized directory of locations.

In some embodiments, the methods comprise requesting medical imaging data for a patient, wherein the patient has an electronic health records (EHR) in a first computer system and patient demographics from the EHR are used to generate a request; receiving from a second computer system one or more sets of exam demographics for the patient, wherein a set of exam demographics includes at least one of: date and time of the medical image data capture, location of the medical image data capture, and description of medical image data; selecting one or more sets of exam demographics for the patient; transmitting a selection of the one or more sets of exam demographics from the first computer system to the second computer system; and receiving, at the first computer system, the medical imaging data for a patient from the second computer system. The methods may further comprise comparing patient demographics with a plurality of existing patients in the second computer system, searching at the second computer system for one or more sets of exam demographics for the patient, and retrieving the medical imaging data for the patient. The methods may further comprise determining at the first computer system a patient identification number for the patient, wherein the request further includes the patient identification number. The methods may further comprise identifying at the second computer system a patient identification number for the patient based on the patient demographics provided with the request. The patient identification number may be obtained from a patient identity system or an Electronic Health Record (EHR) local to the first or second computer system. The exam demographics and/or medical imaging data may be stored on a local server or database integrated with the second computer system.

In some embodiments, the methods comprise entering a medical exam order into a first computer system; transmitting the medical exam order from the first computer system to a medical exam device; and transferring medical exam data from the medical exam device to the first computer system. The methods may further comprise storing the medical exam data on a local server integrated with or connected to the first computer system and/or transferring the medical exam data from the first computer system to a second computer system; and storing the medical exam data on a local server integrated with or connected to the second computer system. In some embodiments, the medical exam request is entered into the first computer system from a user device integrated with the first computer system. In some embodiments, the medical exam device is integrated with the first computer system. In some embodiments, the medical exam device is integrated with a second computer system. In some embodiments, transmitting the medical exam order from the first computer system to the medical exam device comprises: transmitting the medical order from the first computer system to the second computer system; and transmitting the medical order from the second computer system to the medical exam device. In some embodiments, transferring medical exam data from the medical exam device to the first computer system comprises: transferring the medical exam data from the medical exam device to the second computer system; and transferring the medical exam data from the second computer system to the first computer system. The medical exam data may include medical imaging data.

In some embodiments, the methods comprise transmitting from a second computer system one or more sets of medical imaging data, and exam demographics, and patient demographics for a patient, wherein a set of exam demographics includes at least one of: date and time of the medical imaging data capture, location of the medical imaging data capture, and description of medical imaging data; and receiving at a first computer system the one or more sets of medical imaging data, patient demographics, and exam demographics, wherein the patient has an electronic health record (EHR) in the second computer system. In some embodiments, the methods further comprise identifying at the first computer system a patient identification number for the patient by comparing the patient demographics provided with the request demographics with a plurality of existing patients in the first computer system or creating an EHR for the patient in the first computer system with the provided patient demographics. In some embodiments, the methods further comprise adding the one or more sets of medical imaging data, the exam demographics, or a combination thereof to the EHR for the patient. In some embodiments, the methods further comprise selecting the one or more sets of medical imaging data, and exam demographics for the patient at the second computer system. In some embodiments, the methods further comprise storing the one or more sets of medical imaging data and exam demographics on a local server or database integrated with the first computer system.

The medical imaging data or medical exam data may include data objects encoded in Digital Imaging and Communication in Medicine (DICOM) format. The medical imaging data may include data in a non-DICOM format (e.g., JPEG, GIF, PNG, MPG, MP4, or MOV format).

Further disclosed herein are non-transitory computer-readable media storing instructions that when executed by one or more processors perform the computer implemented methods described herein.

Other aspects and embodiments of the disclosure will be apparent in light of the following detailed description and accompanying figures.

### BRIEF DESCRIPTION OF THE DRAWINGS

FIG. 1 is a schematic of an exemplary system for the exchange of medical imaging data.

FIG. 2 is an exemplary workflow for the exchange of medical exam data.

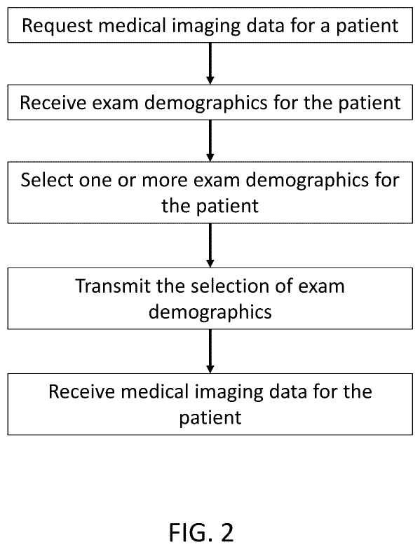

FIG. 3 is a schematic of an exemplary system for medical exam order queuing and routing.

FIG. 4 is a schematic of an exemplary system for medical exam order queuing and routing and the exchange of resulting medical imaging data.

FIG. 5 is a schematic of exemplary workflows for medical exam order queuing and routing and the exchange of resulting medical exam data.

FIG. 6 is an exemplary workflow for the exchange of medical exam data.

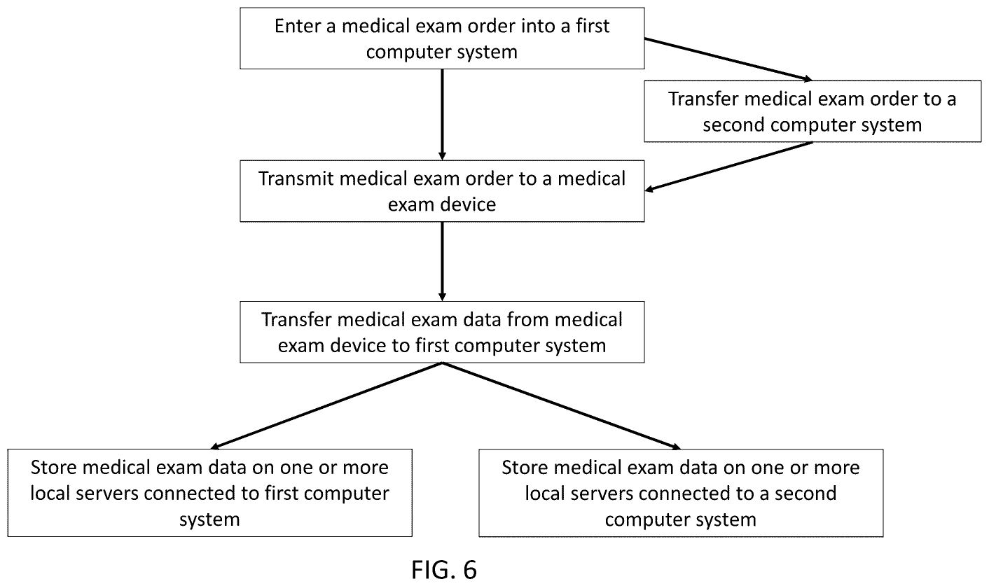

### DETAILED DESCRIPTION

The present disclosure provides methods and systems for image exchange which allow seamless retrieval and transmission of remote imaging studies for one or more off-site locations without additional and cumbersome authentication and minimal user input. The systems and methods are directed to peer-to-peer image transfer through a secure connection rather than a separate cloud server. The computer system is integrated with the EHR and/or system of patient record at the requesting site and, in some embodiments, image transfer is limited to patients presently in the EHR and/or system of patient record.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “patient” may be human or non-human and may include either adults or juveniles (e.g., children). Moreover, patient may mean any living organism, preferably a mammal (e.g., human or non-human). Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The present disclosure provides systems and methods for the exchange of medical data (e.g., medical imaging data between a first computer system and a second computer system). The methods comprise requesting medical imaging data for a patient at a first computer system and receiving, at the first computer system, the medical imaging data for a patient from the second computer system.

Medical imaging data may include data objects encoded in Digital Imaging and Communication in Medicine (DICOM) format. The medical imaging data may also or alternatively include medical imaging data in a non-DICOM format. The medical imaging data can include images in any image format (e.g., JPEG, GIF, PNG, or other image format) or non-image data associated with medical imaging (e.g., text documents describing the medical images or exam demographics). For example, the non-images can include PDF files, video files, non-image data in HL7, CDA, XML formats, or other non-image data in a variety of formats.

The first and second computer systems may be integrated into a single local network connected through a wired or wireless connection (e.g., local enterprise network). The first and second computer systems may be integrated into two different local networks. For example, networks at the same or different physical location (e.g., hospital systems or satellite locations with a single hospital or health system) which may be connected through the internet.

The first computer system and the second computer system may each be connected in a peer to peer network. Peer-to-peer (also referred to as P2P) is a type of Internet network that allows a group of computer systems (peers) to connect with each other and directly access files without an intermediary server, such that each peer computer system can be both a client and a server. In some embodiments, the first computer system determines the address of the second computer system using a centralized directory of locations.

In some embodiments, the files may route through remote or cloud servers.

The first and second computer systems may be integrated with or connected to one or more servers or databases (e.g., directory services server, EHR, imaging archive systems (e.g., picture archiving and communication systems (PACS) or Vendor Neutral Archives (VNA)), secondary processing units or information systems (e.g., cardiovascular information systems)). The first and second computer systems may be integrated with or connected to with one or more user devices (e.g., physician devices, medical imaging devices).

In some embodiments, the methods comprise requesting medical imaging data for a patient, wherein the patient has an EHR in a first computer system and patient demographics from the EHR are used to generate a request. The patient demographics may include a first name, a last name, if applicable, a maiden or previous name, date of birth, sex, or any combination thereof. The first computer system may also determine a patient identification number to be included in the request. The patient identification number may be obtained from a patient identity system (e.g., master patient index (MPI) or enterprise master patient index (eMPI)) integrated into or with the first computer system or the EHR of the patient. Other unique identifiers of the patient may also be included in the request.

The request may be entered into the first computer system from a user device integrated into the first computer system. The user device may be a desktop computer, a laptop, a tablet computer, a smartphone, or similar computer device that is configured to allow integration with the first computer system.

The method comprises receiving from a second computer system one or more sets of exam demographics for the patient, wherein a set of exam demographics includes at least one of: date and time of the imaging data capture, location, and description of imaging data. The second computer system may determine a patient identification number, or other unique identifier for the patient, if not provided in the request. The patient identification number may be obtained from a patient identity system or the local EHR.

The method may also comprise the second computer system comparing the patient demographics from the request with a plurality of existing patients in the second computer system or server databases locally integrated with the second computer system. The second computer system may query exact matches and/or approximate matches (e.g., running fuzzy searches) to identify non-exact matches of potential patients which may be the target patient. The second computer will also search (e.g., DICOM Query procedure) for the selected exam demographics for the identified patients.

Following the selection of one or more sets of exam demographics for the patient at the first computer, the second computer retrieves (e.g., using a DICOM Retrieve procedure) the medical imaging data for the patient from within the computer system or a local server or database integrated with the second computer system and transmits (e.g., using a DICOM Send procedure or a secure non-DICOM mechanism) the medical imaging data for the patient to the first computer system.

The method incorporates a security model based on trust relationships between healthcare organizations (e.g., first and second computer systems). Each individual organization defines other organizations that are trusted for transmission or retrieval of data. For a retrieval, the trust relationship can specify whether or not the patient must exist in the EHR of the requesting organization. For a transmission, the trust relationship can specify whether the patient must exist in the EHR of the receiving organization. When the first computer system connects to the directory service, it receives a list of organizations that will allow a transmission or retrieval of data based on the previously defined security relationships. This method has significant advantages over traditional patient-level trust relationships that require consents, etc. for each patient data transfer.

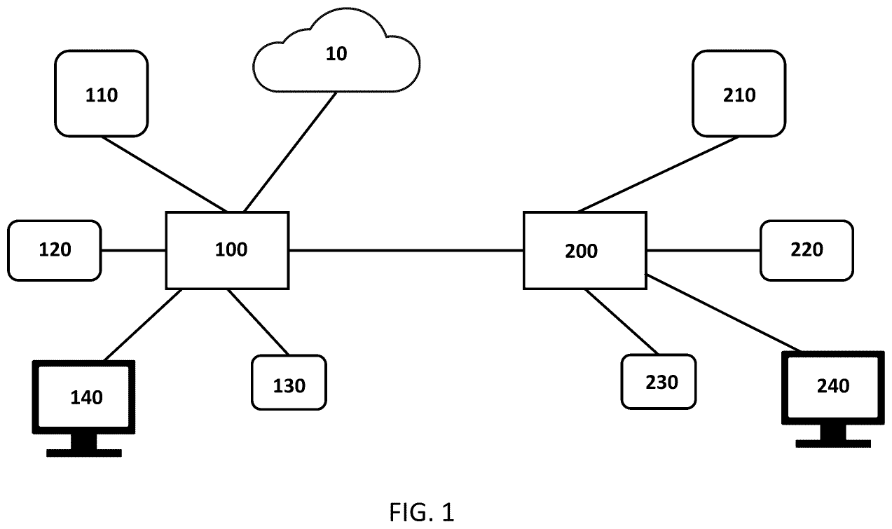

As shown in FIG. 1, first computer system 100 may be integrated with user device 140 from which the EHR can be accessed. Within the EHR, the user enters a request for image transfer into first computer system 100. The request is populated with patient demographics from EHR 120 and, optionally, unique identifier(s) for the patient determined following search of local patient identity system 130. The first computer system 100 communicates with centralized server 10 to find the address of computer system 200. First computer system 100 sends a request for images directly to second computer system 200 through an encrypted, secure channel. The request includes patient demographics including name(s), date of birth, gender of the patient, and optionally unique identifier(s) for the patient. Alternatively, or additionally, second computer system 200 can search for or confirm a unique patient identifier from the patient demographics with local patient identity system 230 or local EHR 220. This confirmation and search ensure that older medical records that may be associated with a previous name for the patient are also retrieved.

Based on the patient demographics and/or the unique identifier(s), second computer system 200 may perform a standard query (e.g., DICOM or non-DICOM Query) of imaging archive 210 (e.g., PACS or VNA) for one or more sets of exam demographics. The retrieved exam demographics are transmitted back through the secure channel to first computer system 100 and may be displayed on user device 140 for selection of the desired medical imaging data for the patient. The request of the desired medical imaging data is transmitted to second computer system 200 which retrieves the exams medical imaging data (e.g., using DICOM Retrieve procedure) from imaging archive 210 and transmits the imaging data (e.g., via a secure channel or with DICOM Send procedure) to first computer system 100, which may save the medical imaging data in imaging archive 110.

Second computer system 200 may send a study comprising one or more sets of medical imaging data, and exam demographics, and patient demographics for a patient to first computer system 100 without a request of transfer from first computer system 100. As shown in FIG. 1, second computer system 200 may be integrated with user device 240 from which the EHR can be accessed. Within the EHR, the second computer system 200 communicates with centralized server 10 to find the address of computer system 100. A user at the second system may select a study to send and then send a study from image archive 210 of second system 200 to first computer system 100. Based on either a query of a patient identity system (e.g., master patient index (MPI) or enterprise master patient index (eMPI)) or for patients with matching demographics, the second computer system 200 may add the study to EHR 120 of the first computer system 100. Alternatively, second computer system 200 may send the study and a user of first computer system 100 associates the study to a patient in local EHR 120 to first computer system 100. If no patient exists in first computer system 100, a patient record may be created based on the patient demographics provided with the medical imaging data. In some embodiments, first computer system 100 may be configured to automatically create a patient record if none is located in the system upon receipt of the medical imaging data.

Second computer system 200 may be integrated with user device 230 from which the EHR can be accessed. Within the EHR, the user enters a request for image transfer to first computer system 100. The request is populated with patient demographics from the EHR and, optionally, unique identifier(s) for the patient determined following search of local patient identity system 230. Second computer system 200 communicates with centralized server 10 to find the address of computer system 100. Second computer system 200 sends medical imaging data directly to first computer system 100 through an encrypted, secure channel. The medical imaging data may include patient demographics including name(s), date of birth, gender of the patient, and optionally unique identifier(s) for the patient. Alternatively, or additionally, first computer system 100 can search for or confirm a unique patient identifier from the patient demographics with local patient identity system 130 or local EHR 120 upon receipt or at a time following receipt of the medical imaging data. The medical imaging data may be transferred from first computer system 100 to an imaging archive 110, and accessible from user device 140 from which the local EHR can also be accessed.

The present disclosure also provides systems and methods for the exchange of medical data (e.g., medical imaging data) as a result of a medical exam order. The systems and methods comprise entering a medical exam order into a first computer system and transmitting the medical exam order from the first computer system to a medical exam device. The medical exam order may be entered into the first computer system, for example from an integrated user device, weeks in advance of the date of the medical exam and then transferred to the medical exam device the day of the medical exam. Medical exam devices may include EKG carts, CT scanners, MRI scanners, x-ray image processors, ultrasound machines, image processing workstations, and the like. The systems and methods further comprise transferring medical exam data from the medical exam device to the first computer system.

In some embodiments, the systems and methods comprise transferring or storing data from the medical exam to one or more local servers or databases (e.g., directory services server, EHR, imaging archive servers (e.g., picture archiving and communication systems (PACS)), secondary processing units or information systems (e.g., cardiovascular information systems)) connected to or integrated with the first computer system. Alternatively, or additionally, the systems and methods comprise transferring the medical exam data from the first computer system to a second computer system and storing the medical exam data on one or more local servers or databases connected to or integrated with the second computer system.

The medical exam device may be integrated with the first computer system or the second computer system. In some embodiments, transmitting the medical exam order from the first computer system to the medical exam device comprises transmitting the medical order from the first computer system to the second computer system and transmitting the medical order from the second computer system to the medical exam device. In some embodiments, transferring medical exam data from the medical exam device to the first computer system comprises transferring the medical exam data from the medical exam device to the second computer system and transferring the medical exam data from the second computer system to the first computer system.

The first computer system and the second computer system may be connected in a peer-to-peer network. In some embodiments, the first computer system or the second computer system may determine the address of the other computer system using a centralized directory of locations.

The medical exam data may include medical imaging data. The medical imaging data may include data objects encoded in Digital Imaging and Communication in Medicine (DICOM) format. The medical imaging data may also or alternatively include medical imaging data in a non-DICOM format. The medical imaging data can include images in any image format (e.g., JPEG, GIF, PNG, or other image format) or non-image data associated with medical imaging (e.g., text documents describing the medical images or exam demographics). For example, the non-images can include PDF files, video files, non-image data in HL7, CDA, XML formats, or other non-image data in a variety of formats.

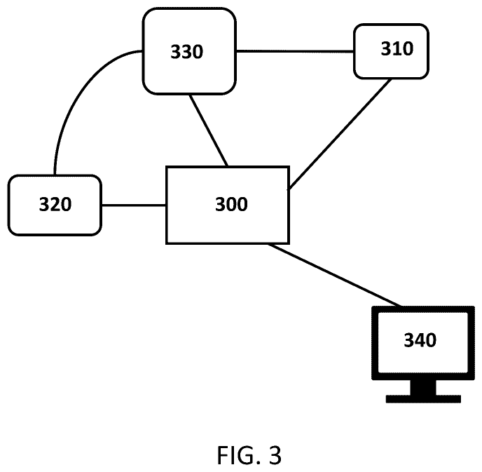

FIG. 3 is a schematic of an example of medical exam order queuing in which the medical exam device 340 is integrated with the first computer system 300. In this example, an order for a procedure may be entered into EHR 320 integrated with first computer system 300. Medical exam devices commonly only support order entry on the day of the exam, but the order is likely entered into the first computer system 300 in advance of the medical exam day. Computer system 300 maintains the order queue for the medical exam device 340. On the appropriate date, computer system 300 transmits one or more orders to medical exam device 340. Following the medical exam, medical exam data is transmitted back to first computer system 300. First computer system 300 may then route or save the medical exam data to a server or database connected with the first computer system, for example a secondary processing system 330 (e.g., Cardiology Information System) or image archive 310 (e.g., PACS). The secondary processing system may route the medical exam results to image archive 310 or the EHR 320.

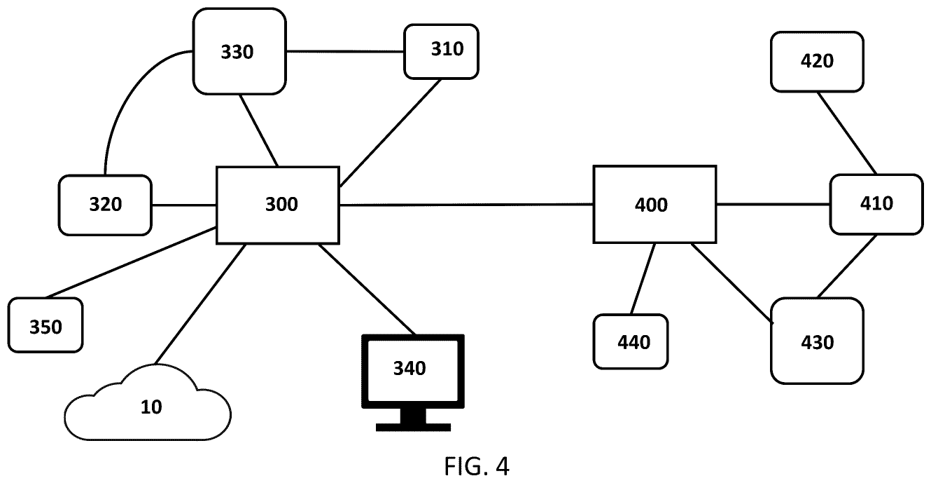

The medical exam results may be transmitted to a second computer system. For example, as shown in FIG. 4 first computer system 300 is at one health system and second computer system 400 is located at a second health system. The medical exam results may be routed through a secure, proprietary HTTPS connection to second computer system 400. The second computer system 400 may then route or save the medical exam data to a server or database connected with the first computer system, for example a secondary processing system 410 (e.g., Cardiology Information System) or image archive 430 (e.g., PACS). The secondary processing system may route the medical exam results to image archive 430 or the EHR 420. Optionally, when the results are received, a unique patient identifier may be assigned to the medical exam results from patient identity system 440 integrated with second computer system 400. First computer system 300 may add a unique patient identifier to the medical exam order from patient identity system 350. In addition, first computer system 300 may determine the location of second computer system 400 from centralized directory 10.

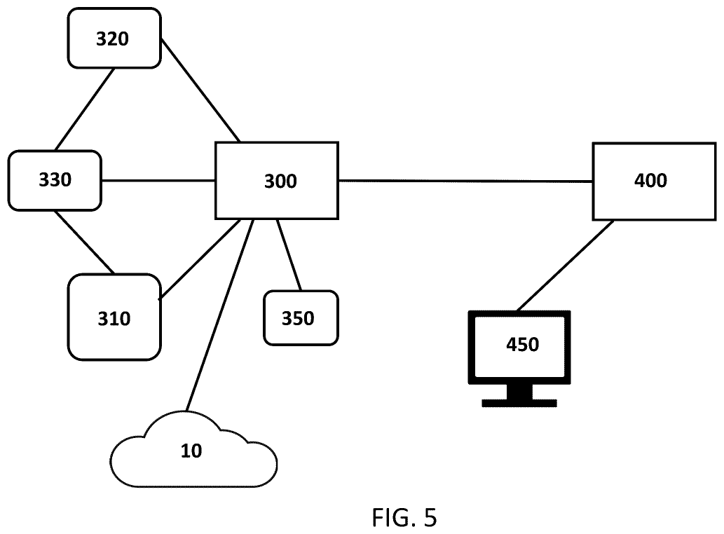

Shown in FIG. 5 is a schematic of an example of remote medical exam order queuing in which medical exam device 450 is integrated with second computer system 400. This is commonly encountered when a medical exam device is shared between health systems and the results need to be routed back to the requesting system. In this example, first computer system 300 has an order for a medical exam on medical exam device 450 integrated with second computer system 400. The medical exam order is routed to medical exam device 450 from first computer system 300 through second computer system 400. The medical exam order may be transferred to second computer system 400 in advance of the day of the medical exam. Second computer system 400 adds the medical exam order to the queue and, on the day of the medical exam, sends the order to medical device 450. Following the medical exam, medical exam data is transmitted back to second computer system 400. Second computer system 400 transmits the medical exam results through a secure, proprietary HTTPS connection to first computer system 300. The first computer system 300 may then route or save the medical exam data to a server connected with the first computer system, for example a secondary processing system 330 (e.g., Cardiology Information System) or image archive 310 (e.g., PACS). The secondary processing system may route the medical exam results to image archive 310 or the EHR 320. Optionally, when the results are received, a unique patient identifier may be assigned to the medical exam results from patient identity system 350.

The methods described herein can be implemented in digital electronic circuitry, or in computer software, firmware, or hardware. The methods can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated, propagated signal, for example, a machine-generated electrical, optical, or electromagnetic signal that is generated to encode information for transmission to suitable receiver apparatus for execution by a data processing apparatus. In some embodiments, the methods are implemented as a non-transitory computer-readable medium storing instructions executable by one or more processors to perform operations.

The present disclosure also provides non-transitory computer-readable media. The non-transitory computer-readable media stores instructions that when executed by one or more processors performs some or all of the operations described in the disclosed methods.

In some embodiments, the one or more processors perform operations comprising at least one or all of: requesting medical imaging data for a patient, wherein the patient is in the electronic health records (EHR) of a first computer system and patient demographics from the EHR are used to generate a request; receiving from a second computer system one or more sets of exam demographics for the patient, wherein a set of exam demographics includes at least one of: date and time of the imaging data capture, location one or more sets of exam demographics of the imaging data capture, and description of imaging data; selecting one or more sets of exam demographics for the patient; transmitting a selection of the one or more sets of exam demographics from the first computer system to the second computer system; and receiving, at the first computer system, the medical imaging data for a patient from the second computer system. The processors may further perform any or all of the operations of: determining a patient identification number for the patient, wherein the request further includes the patient identification number; identifying at the second computer system a patient identification number for the patient based on the patient demographics provided with the request; comparing patient demographics with a plurality of existing patients in the second computer system; identifying a potential match for the patient from the plurality of existing patients; searching at the second computer system for one or more sets of exam demographics for the patient; and retrieving the medical imaging data for the patient.

In some embodiments, the one or more processors perform operations comprising at least one or all of: entering a medical exam order into a first computer system; transmitting the medical exam order from the first computer system to a medical exam device; and transferring medical exam data from the medical exam device to the first computer system. The processors may further perform any or all of the operations of: storing the medical exam data one or more local servers integrated within the first computer system; transferring the medical exam data from the first computer system to a second computer system; storing the medical exam data one or more local servers integrated within the client computer system; transmitting the medical order from the first computer system to the second computer system; transmitting the medical order from the second computer system to the medical exam device; transferring the medical exam data from the medical exam device to the second computer system; and transferring the medical exam data from the second computer system to the first computer system.

The methods described herein can be implemented as a system including one or more processors and a computer-readable medium storing instructions executable by the one or more processors to perform operations, as described above. The system may comprise at least one computer system comprising the one or more processors and/or the computer-readable media. The system may further comprise one or more local servers or databases connected to or integrated with the one or more computer system. The local servers or databases may include a directory services server, an EHR, imaging archive servers (e.g., picture archiving and communication systems (PACS)), secondary processing units or information systems (e.g., cardiovascular information systems)), and the like. The system may further comprise one or more devices integrated with the computer systems. For example, the devices may include a user device configure to allow a user (e.g., a clinician) to access and use the computer system. The devices may also include medical exam devices, including, for example EKG carts, CT scanners, MRI scanners, x-ray image processors, ultrasound machines, image processing workstations, and the like.

The systems, methods and readable media described herein protect the confidentiality and security of protected health information (PHI) in compliance with various patient privacy standards (e.g., Health Insurance Portability and Accountability Act (HIPAA)). Thus, the systems, methods and readable media may be considered HIPAA-compliant. The systems, methods and readable media may provide or allow one or all of: means of access control, mechanisms to authenticate electronic PHI, functionalities for encryption/decryption, and mechanisms to log activity and implement audits.

Data may be communicated using known encryption/decryption and security techniques. For example, DICOM imaging standards support encryption. User authentication at the request site, for example with integration with the EHR for the patient, provides access controls and auto-logoff functionality, as well as restricts transfer of images to patient known in the local EHR. Peer-to-peer transfers eliminate exposure of data residing in the cloud, like many commercially available systems, and the computer systems are maintained in secure local environments with access and intrusion security protections in place. The system and methods may anonymize the patient before data sharing or provide patient info as a separate message, with a security code or token to match up the medical exam data to further verify the selected patient is correct.

1. A computer-implemented method comprising:
requesting medical imaging data for a patient, wherein the patient has an electronic health record (EHR) in a first computer system and patient demographics from the EHR are used to generate a request;
receiving from a second computer system one or more sets of exam demographics for the patient, wherein a set of exam demographics includes at least one of: date and time of the medical imaging data capture, location of the medical imaging data capture, and description of medical imaging data;
selecting one or more sets of exam demographics for the patient;
transmitting a selection of the one or more sets of exam demographics from the first computer system to the second computer system; and
receiving, at the first computer system, the medical imaging data for the patient from the second computer system.

2. The method of claim 1, further comprising one or both of:
determining at the first computer system a patient identification number for the patient, wherein the request further includes the patient identification number; and
identifying at the second computer system a patient identification number for the patient based on the patient demographics provided with the request.

3. (canceled)

4. The method of claim 2, wherein the patient identification number is obtained from a patient identity system or an Electronic Health Record local to the first or second computer system.

5. The method of claim 1, further comprising one or more of:
comparing patient demographics with a plurality of existing patients in the second computer system;
searching at the second computer system for one or more sets of exam demographics for the patient;
identifying a potential match for the patient from the plurality of existing patients;
retrieving the medical imaging data for the patient; and
transmitting the medical imaging data to the first computer system.

6. (canceled)

7. The method of claim 1, wherein the exam demographics and/or medical imaging data are stored on a local server or database integrated with the second computer system.

8. The method of claim 1, wherein the medical imaging data includes data objects encoded in Digital Imaging and Communication in Medicine (DICOM) format.

9. The method of claim 5, wherein the searching uses a DICOM Query procedure.

10. The method of claim 5, wherein the medical imaging data is retrieved by a DICOM Retrieve procedure.

11. (canceled)

12. The method of claim 1, wherein the patient demographics comprise: first name, last name, maiden or previous name, date of birth, sex, or any combination thereof.

13. The method of claim 1, wherein the first computer system determines a location or address of the second computer system using a centralized directory of locations.

14. The method of claim 1, wherein the request is entered into the first computer system from a user device integrated with the first computer system.

15. The method of claim 1, wherein the first computer system and the second computer system are connected in a peer to peer network.

16. A computer-implemented method comprising:
entering a medical exam order into a first computer system;
transmitting the medical exam order from the first computer system to a medical exam device; and
transferring medical exam data from the medical exam device to the first computer system.

17. The method of claim 16, further comprising one or more of:
storing the medical exam data on one or more local servers connected to the first computer system;
transferring the medical exam data from the first computer system to a second computer system; and
storing the medical exam data on one or more local servers connected to the second computer system.

18. (canceled)

19. The method of claim 16, wherein the request is entered into the first computer system from a user device integrated with the first computer system.

20. The method of claim 17, wherein the medical exam device is integrated with the first computer system or the second computer system.

21. (canceled)

22. The method of claim 20, wherein transmitting the medical exam order from the first computer system to the medical exam device comprises:
transmitting the medical order from the first computer system to the second computer system; and
transmitting the medical order from the second computer system to the medical exam device.

23. The method of claim 20, wherein transferring medical exam data from the medical exam device to the first computer system comprises:
transferring the medical exam data from the medical exam device to the second computer system; and
transferring the medical exam data from the second computer system to the first computer system.

24. The method of claim 17, wherein the first computer system and the second computer system are connected in a peer to peer network and wherein the first computer system and the second computer system determine a location or address of the other computer system using a centralized directory of locations.

25. (canceled)

26. The method of claim 16, wherein the medical exam data includes medical imaging data.

27-54. (canceled)
